(19) United States
(12) Patent Application Publication
(10) Pub. No.: US 2002/0071828 A1
(43) Pub. Date: Jun. 13, 2002

(54) METHOD OF STIMULATING NERVE REGENERATION

(76) Inventors: PASCAL PEULVE, NOTRE DAME DE BONDEVILLE (FR); FREDERIC REVAH, PARIS (FR); MARC TADIE, PARIS (FR)

Correspondence Address: FINNEGAN HENDERSON FARABOW, GARRETT & DUNNER, 1300 I STREET N W, WASHINGTON, DC, 20005-3315, US

(21) Appl. No.: 09/401,319

(22) Filed: Sep. 23, 1999

Related U.S. Application Data
(63) Continuation of application No. PCT/FR98/00595, filed on Mar. 25, 1998.

(30) Foreign Application Priority Data
Mar. 26, 1997 (FR) 97/03,672

(51) Int. Cl. A61K 48/00; C07H 21/04

(52) U.S. Cl. 424/93.2; 424/93.6; 514/44; 435/320.1; 435/325; 536/23.1

(57) ABSTRACT

A method used to stimulate neural regeneration can be applied to peripheral nerves as well as nerves of the central nervous system, especially of the spinal cord. The invention notably employs a system including a biocompatible cuff, into which a system of expression of a neurotrophic factor is inserted.

The present invention relates to the field of the biology, and in particular of the medical biology, of the nervous system. It relates more particularly to the methods of stimulating nerve regeneration which are applicable both to the peripheral nerves and in the central system, and in particular the spinal cord. By virtue of their local and specific character, the methods of the invention can be used to stimulate nerve regeneration in various pathological situations, and in particular in cases of lesions of the spinal cord, of peripheral nerves, or of the brachial or lumbar plexus.

Lesions of the nervous system, both central (CNS) and peripheral (PNS), are frequent in traumatology and are serious. Thus, medullary lesions, whether of traumatic or degenerative origin, peripheral nerve lesions, or brachial or lumbar plexus lesions leave up until now the injured or sick individuals seriously handicapped for life. Although the PNS has a high capacity to regenerate spontaneously, the use of conventional nerve repair techniques gives only disappointing results. These techniques mainly consist of direct anastomosis or the fitting of an autologous or heterologous nerve graft when the tensions are too high to allow suture of the two nerve endings (in case of loss of substance, or of excessive laceration of the nerve requiring resection of a nerve segment). With these techniques, less than 5% of the patients who have had a median nerve repair in the wrist rediscover a normal sensation or motor function after 5 years(1).

More recently, the use of tubular prostheses joining the ends of an injured nerve (cuffing technique) has offered an alternative to these conventional nerve repair techniques (2,3). This technique offers the advantage of simplifying the conditions for realigning the nerve bundles, and has made it possible to successfully bridge, both experimentally and also clinically, small losses of substance (up to 5-7 mm (1-6)). However, it appears that in order to bridge losses of substance of a larger size, the addition of neurotrophic substances or of cells inside the tubes is essential. Experimentally, several factors have been tested, such as FGF-1 and -2, or NGF as an in vivo application in the stent (7-10), or CNTF or IGF II in systemic application, without, as a result, clearly demonstrating their role in nerve regeneration (7-12). Moreover, no clinical treatment currently exists for spinal cord lesions.

The present invention provides a solution to this problem of treating nerve, traumatic or degenerative lesions. The present invention relates, indeed, to a method of stimulating nerve regeneration by means of a biocompatible cuff and of a composition of nucleic acids encoding neurotrophic factors. The present invention also relates to a device for stimulating nerve regeneration, comprising a biocompatible cuff into which a system for expressing a nerve growth stimulating factor (neurotrophic factor) is introduced. Another aspect of the invention relates to a kit for stimulating nerve regeneration, comprising, on the one hand, a biocompatible cuff, and, on the other hand, a composition comprising a system for expressing a nerve growth stimulating factor. The present invention also relates to the use, for the preparation of a composition intended to stimulate nerve regeneration, of a biocompatible cuff into which a system for expressing a neurotrophic factor is introduced.

The present invention is, in addition, applicable to both the regeneration of the peripheral nerves and for stimulating axonal regeneration in the spinal cord.

The present invention results from several observations. It results in particular from the demonstration that it is possible to surgically establish a physical bridge between two sections of a nerve by means of an appropriate device, and to introduce a system for expressing a neurotrophic factor into this device. It also results from the demonstration that it is possible to induce a local concentration of trophic factors, for a sufficient duration to stimulate neuronal growth. The present invention thus combines several properties which are particularly advantageous from the therapeutic point of view. It allows, first of all, a lasting action, by virtue of an effect of prolonged release of the trophic factor. The biological effect of the neurotrophic factor is, furthermore, potentiated by the stent effect of the cuff which makes it possible to accelerate and to guide neuronal growth. The method of the invention also allows a very local, and therefore very specific, action, the trophic factors being enclosed in a sealed device, on the site of the trauma or of the degeneration. The results presented in the examples show, to this end, that the method of the invention allows a rapid, effective and local repair of nerves.

The method of the invention consists, more particularly, in acting locally at the level of a nerve section. The proximal or distal section of the sectioned nerve or bundle is introduced at one end of a biocompatible cuff, where it is physically kept in place. A composition comprising a system for expressing a neurotrophic factor is then introduced into the said cuff. The second section of the sectioned nerve or bundle is then introduced at the other end of the cuff, where it is also physically kept in place. To avoid diffusion of the expression system outside the cuff, it is advantageously ligated and/or held with a biological glue at the ends. This device can, in addition, allow new injections of expression systems. The presence both of the support and of the neurotrophic factor in high concentration and for a prolonged period makes it possible, as illustrated in the examples, to reconstitute nerve continuity and, thus, to restore the corresponding activity.

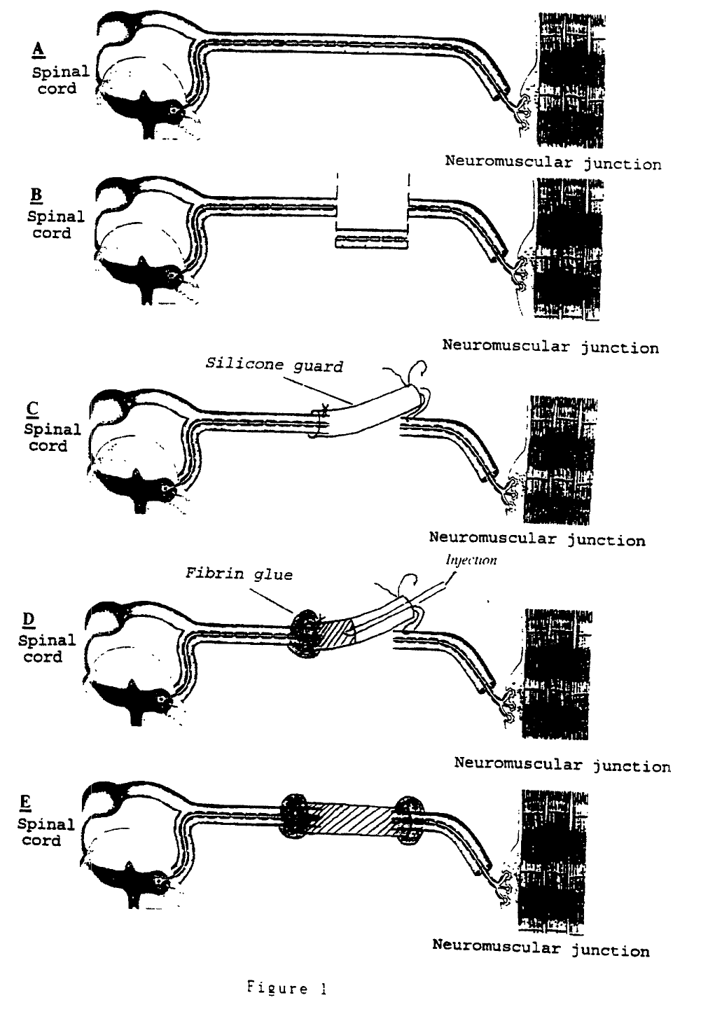

In a manner which is more specific to the peripheral nervous system, the method of the invention consists in taking a peripheral nerve or a root which is subjacent to the lesion and fitting a biocompatible cuff after resection of part of the nerve or of the root. The proximal part of the section, whether it has motor function or is sensitive, is introduced into the cuff and held in place, for example, by a suture or by introducing a biological glue. The expression system encoding the active factor is injected into this cuff, which is left in place. The distal part of the section is then reconnected to the other end of the cuff, allowing the restoration of axonal continuity (FIG. 1).

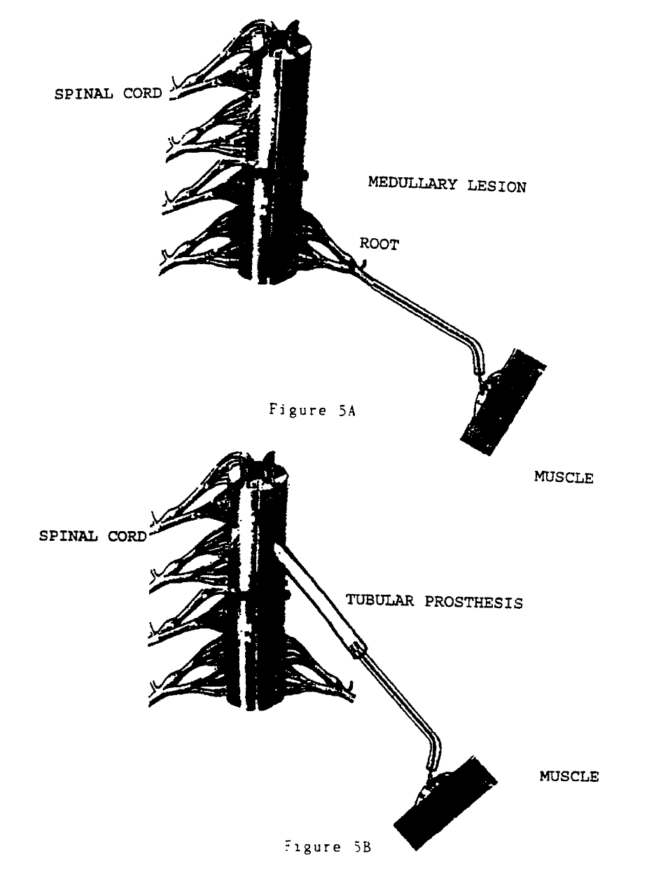

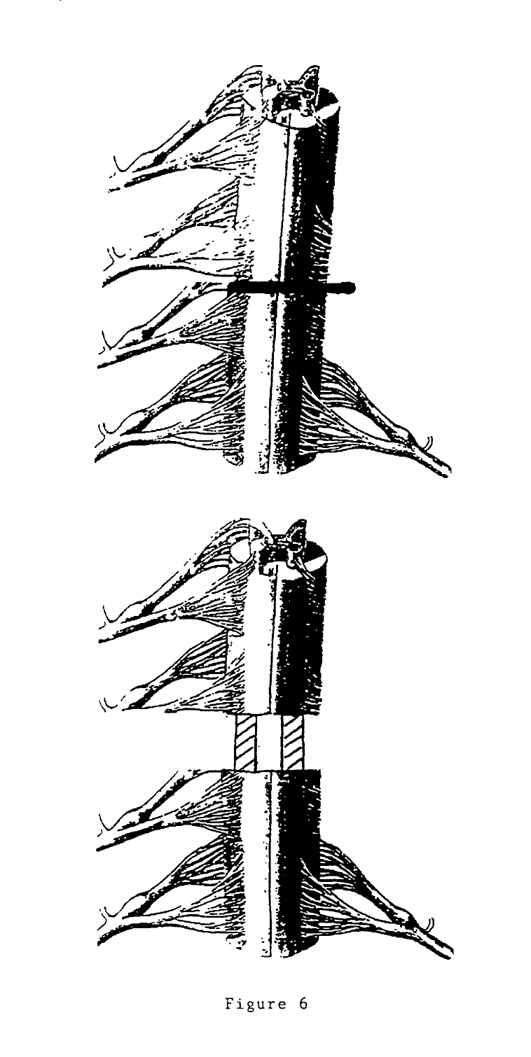

At the level of the central, and in particular medullary, nervous system, the method of the invention is also particularly suitable for bridging lesions in the spinal cord. This type of trauma moreover constitutes one of the main applications of the system of the invention, and for which no clinical treatment exists up until now. Two types of applications may be envisaged: either the bridging of the peripheral afferences subjacent to a lesion to the healthy marrow superjacent to this lesion (FIG. 5), or the bridging of the healthy marrow superjacent to a lesion, to the marrow subjacent to this lesion (FIG. 6).

In the first case, one or more roots subjacent to a medullary lesion (FIG. 5A) are sectioned, introduced into a tubular prosthesis (cuff) and held in place with the aid of sutures or of a biological glue. The stent can then receive expression systems carrying genes capable of stimulating axonal elongation of the motoneurons; and/or, optionally, various factors known to stimulate axonal regrowth such as a peripheral nerve graft, or cells. The stent is then introduced into a longitudinal incision made in the healthy marrow superjacent to the lesion so that the proximal end of the tube touches the anterior horn of the grey matter (site of location of the spinal motoneurons). The stent is attached with one or more sutures to the arachnoid and biological glue (FIG. 5B). Such an assembly can thus return functionality to certain key muscles.

In the second case, the injured part of the marrow is excised and a stent is implanted upstream and downstream so as to join the main bundles (pyramidal, corticospinal and the like) between the parts above and below the injured parts (FIG. 6). The stent is then filled with the same substances as above.

Within the framework of the invention, proximal part of the section or proximal part of the nerve is understood to mean the part of the nerve which is in contact with the central nervous system. In the case of a peripheral nerve, its proximal part is that which is connected to the spinal cord. In the case of a lesion of the spinal cord, the proximal part is that which is in contact with the central nervous system.

Distal part of the section, or distal part of the nerve, is also understood to mean the peripheral part of the nerve. In the case of a peripheral nerve, its distal part is therefore that which is connected to the motor endplate (neuromuscular junction). In the case of a lesion of the spinal cord, the distal part is that which becomes disconnected from the central nervous system.

To carry out the invention, the cuff may consist of any device which is compatible with a therapeutic use. The structure and the composition of the cuff are advantageously defined such that (i) it restores axonal continuity, (ii) it can contain a composition comprising a system for expressing active factors, (iii) it can serve as stent for axonal regrowth, from the spinal cord towards the periphery, from the periphery towards the spinal cord, and from the spinal cord towards the spinal cord. The stent property of the cuff exerts itself through the ability of the nerves to adhere and to grow on it, in particular on its inner face. The adherence may result from any form of biological and/or chemical and/or physical interaction causing the adhesion and/or the attachment of the cells on the cuff. Moreover, for applications in human therapy, it is also desirable that the cuff is of the impermeable or semipermeable type, but not allowing the passage of the expression system.

Advantageously, the cuff is a solid, nontoxic and biocompatible support. It may be in particular a cuff consisting of synthetic material(s), such as silicone, PAN/PVC, PVFD, polytetrafluoroethylene (PTFE) fibres or acrylic copolymers. In a specific embodiment of the invention, the use of a cuff consisting of or based on biomaterials, such as in particular cross-linked collagen, bone powder, carbohydrate-based polymers, polyglycolic/polylactic acid derivatives, hyaluronic acid esters, or chalk-based supports, is preferred. Preferably, collagen or silicone is used within the framework of the present invention. It may be collagen of human, bovine or murine origin. More preferably, a cuff consisting of a bilayer of type I or III or IV, advantageously IV/IVox, collagen, or of silicone, is used. There may be mentioned, by way of a specific example, a Silastic cuff (Dow-Corning), consisting of silicone. Moreover, the cuff has advantageously a tubular shape, of cylindrical or angular section. The diameter of the cuff can be adjusted by persons skilled in the art according to the desired applications. In particular, for stimulating the regeneration of a peripheral nerve, a relatively small diameter, from 0.05 to 15 mm, can be used. More preferably, the inner diameter of the cuff is between 0.5 and 10 mm. For spinal cord regeneration applications, cuffs with a larger inner diameter can be chosen. In particular, for these applications, the cuffs used have an inner diameter which may be as high as 15 to 20 mm, depending on the relevant nerve section. For bridging a root avulsed at the level of the brachial plexus, the diameter of the cuff advantageously corresponds to the diameter of the root. The length of the cuff is generally determined by the size of the loss of substance to be compensated for. Cuffs with a length of between 0.5 and 5 cm can be used. Preferably, the length of the cuff remains less than 5 cm, losses of substance greater than 5 cm being less frequent.

As indicated above, the method of the invention consists, in a first instance, in introducing a first part of the nerve into the cuff. This is advantageously the proximal part of the nerve. It is then held in place to ensure (i) a good nerve growth and (ii) a leaktightness of the device. To do this, it is possible to perform a suture between the nerve and the cuff and/or to introduce a biological glue. The suture can be made according to conventional surgical methods using the appropriate thread. The biological glue may be any biocompatible glue, which can be applied to the nervous system. It may be in particular any biological glue used in human surgery, and in particular a glue consisting of fibrin: Biocolle (Biotransfusion, CRTS, Lille), Tissucol (Immuno AG, Vienna, Austria), and the like.

The method of the invention comprises, as indicated above, the introduction, into the cuff, of a composition comprising a system for expressing neurotrophic factors.

For the purposes of the invention, the term “expression system” designates any construct allowing the in vivo expression of a nucleic acid encoding a neurotrophic factor. Advantageously, the expression system comprises a nucleic acid encoding a neurotrophic factor under the control of a transcriptional promoter (expression cassette). This nucleic acid may be a DNA or an RNA. In the case of a DNA, there may be used a cDNA, a gDNA or a hybrid DNA, that is to say a DNA containing one or more introns of the gDNA, but not all. The DNA may also be synthetic or semisynthetic, and in particular a DNA artificially synthesized to optimize the codons or to create reduced forms.

The transcriptional promoter may be any promoter which is functional in a mammalian cell, preferably a human cell, and in particular a nerve cell. It may be the promoter region which is naturally responsible for the expression of the neurotrophic factor considered when it is capable of functioning in the relevant cell or organism. It may also represent regions of different origin (which are responsible for the expression of other proteins, or even synthetic). In particular, it may represent promoter regions of eukaryotic or viral genes. For example, it may represent promoter regions derived from the genome of the target cell. Among the eukaryotic promoters, there may be used any promoter or derived sequence stimulating or repressing the transcription of a gene specifically or otherwise, inducibly or otherwise, strongly or weakly. They may be in particular ubiquitous promoters (promoters of the HPRT, PGK, α-actin or tubulin genes, and the like), promoters of the intermediate filaments (promoter of the GFAP, desmin, vimentin, neurofilament or keratin genes, and the like) promoters of therapeutic genes (for example the promoter of the MDR, CFTR, Factor VIII or ApoAI genes, and the like) or alternatively promoters responding to a stimulus (receptor for steroid hormones, receptor for retinoic acid and the like). Likewise, they may be promoter sequences derived from the genome of a virus, such as for example the promoters of the adenovirus ElA and MLP genes, the CMV early promoter, or alternatively the RSV LTR promoter, and the like. In addition, these promoter regions can be modified by the addition of activating or regulatory sequences or of sequences allowing tissue-specific or predominant expression.

A constitutive eukaryotic or viral promoter is advantageously used within the framework of the invention. This is more particularly a promoter chosen from the promoter of the HPRT, PGK, α-actin or tubulin genes or the promoter of the adenovirus ElA and MLP genes, the CMV early promoter, or alternatively the RSV LTR promoter.

Moreover, the expression cassette advantageously comprises a signal sequence directing the product synthesized in the secretory pathways of the target cell. This signal sequence may be the natural signal sequence of the product synthesized, but it may also be any other functional signal sequence, or an artificial signal sequence.

Finally, the expression cassette generally comprises a region situated in 3′, which specifies a transcriptional termination signal and a polyadenylation site.

The trophic factors which can be used within the framework of the invention are classified essentially in the neurotrophin family, the neurokine family, the beta-TGF family, the fibroblast growth factor (FGF) and insulin-type growth factor (IGF) family (review 16).

More preferably, in the neurotrophin family, the use of BDNF, NT-3 or NT-4/5 is preferred within the framework of the invention.

The brain-derived neurotrophic factor (BDNF), described by Thoenen (17) is a protein of 118 amino acids and with a molecular weight of 13.5 kD. In vitro, BDNF stimulates the formation of neurites and the survival, in culture, of the ganglionic neurons of the retina, of the cholinergic neurons of the septum as well as of the dopaminergic neurons of the mesencephalon (review 18). The DNA sequence encoding the human BDNF and the rat BDNF has been cloned and sequenced (19), as well as in particular the sequence encoding pig BDNF (20). Although its properties are potentially advantageous, the therapeutic application of BDNF is faced with various obstacles. In particular the absence of bioavailability of BDNF limits any therapeutic use. The brain-derived neurotrophic factor (BDNF) produced within the framework of the present invention may be the human BDNF or an animal BDNF.

Neurotrophin-3 (NT3) is a secreted protein of 119 aa which allows the in vitro survival of neurons even at very low concentrations (21). The CDNA sequence encoding human NT3 has been described (22).

The TGF-B family comprises in particular the glial cell derived neurotrophic factor. The glial cell derived neurotrophic factor, GDNF (23) is a protein of 134 amino acids and with a molecular weight of 16 kD. It has the essential capacity of promoting, in vitro, the survival of the dopaminergic neurons and of the motoneurons (16). The glial cell-derived neurotrophic factor (GDNF) produced within the framework of the present invention may be the human GDNF or an animal GDNF. The cDNA sequences encoding the human GDNF and the rat GDNF have been cloned and sequenced (23).

Another neurotrophic factor which can be used within the framework of the present invention is in particular CNTF (“Ciliary NeuroTrophic Factor”). CNTF is a neurokine capable of preventing the death of the neurons. As indicated above, clinical trials were interrupted prematurely for lack of results. The invention now allows the prolonged and continuous in vivo production of CNTF, alone or in combination with other trophic factors. The cDNA and the gene for human and murine CNTF have been cloned and sequenced (EP 385 060; WO 91/04316).

Other neurotrophic factors which can be used within the framework of the present invention are for example IGF-1 (Lewis et al., 1993) and fibroblast growth factors (FGFα, FGFβ). In particular, IGF-I and FGFα are very useful candidates. The sequence of the FGFα gene has been described in the literature, as well as vectors allowing its expression in vivo (WO 95/25803).

Preferably, the expression system of the invention therefore allows the in vivo production of a neurotrophic factor chosen from neurotrophins, neurokines and TGFs. It is more preferably a factor chosen from BDNF, GDNF, CNTF, NT3, FGFα and IGF-I. Of most particular interest is the production of NT3.

Moreover, according to one variant of the invention, it is also possible to use an expression system allowing the production of two neurotrophic factors. In this embodiment, the expression system comprises either two expression cassettes, or a single cassette allowing the simultaneous expression of two nucleic acids (bicistronic unit). When the system comprises two expression cassettes, these may use identical or different promoters.

In the expression systems of the invention, the expression cassette(s) are advantageously part of a vector. This may be in particular a viral or plasmid vector. In the case of an expression system comprising several expression cassettes, the cassettes may be carried by separate vectors, or by the same vector.

The vector used may be a standard plasmid vector, containing, in addition to the expression cassette(s) according to the invention, a replication origin and a marker gene. Various types of improved vectors have moreover been described, free of a marker gene and of a replication origin (WO 96/26270) or possessing, for example, a conditional replication origin (PCT/FR 96/01414). These vectors can be advantageously used within the framework of the present invention.

The vector used may also be a viral vector. Various vectors have been constructed from viruses, which have remarkable gene transfer properties. There may be mentioned more particularly adenoviruses, retroviruses, AAVs and the herpesvirus. For their use as gene transfer vectors, the genome of these viruses is modified so as to make them incapable of autonomous replication in a cell. These viruses are said to be defective for replication. In general, the genome is modified by substitution of the regions essential in trans for viral replication with the expression cassette(s).

Within the framework of the invention, the use of a viral vector derived from adenoviruses is preferred. Adenoviruses are viruses with a linear double-stranded DNA of about 36 (kilobases) kb in size. Their genome comprises in particular an inverted terminal repeat (ITR) at each end, an encapsidation sequence (Psi), early genes and late genes. The main early genes are contained in the E1, E2, E3 and E4 regions. Among these, the genes contained in the E1 region in particular are necessary for viral propagation. The main late genes are contained in the L1 to L5 regions. The genome of the adenovirus Ad5 has been fully sequenced and is accessible on a database (see in particular Genebank M73260). Likewise, parts, or even the entirety, of other adenoviral genomes (Ad2, Ad7, Ad12, and the like) have also been sequenced.

For their use as gene transfer vectors, various adenovirus-derived constructs have been prepared, incorporating various therapeutic genes. More particularly, the constructs described in the prior art are adenoviruses deleted of the E1 region, which is essential for viral replication, into which the heterologous DNA sequences are inserted (Levrero et al., Gene 101 (1991) 195; Gosh-Choudhury et al., Gene 50 (1986) 161). Moreover, to enhance the properties of the vector, it has been proposed to create other deletions or modifications in the adenovirus genome. Thus, a heat-sensitive point mutation was introduced into the mutant ts125, which makes it possible to inactivate the 72 kDa DNA-binding protein (DBP) (13). Other vectors comprise a deletion of another region which is essential for viral replication and/or propagation, the E4 region. The E4 region is indeed involved in the regulation of the expression of the late genes, in the stability of the late nuclear RNAs, in the suppression of the expression of the proteins of the host cell and in the efficiency of the replication of the viral DNA. Adenoviral vectors in which the E1 and E4 regions are deleted therefore possess a transcriptional background noise and a viral gene expression which are highly reduced. Such vectors have been described, for example, in applications WO 94/28152, WO 95/02697 and WO 96/22378. In addition, vectors carrying a modification in the IVa2 gene have also been described (WO 96/10088).

The recombinant adenoviruses described in the literature are produced from various adenovirus serotypes. There are, indeed, various adenovirus serotypes whose structure and properties vary somewhat, but which have a comparable genetic organization. More particularly, the recombinant adenoviruses may be of human or animal origin. As regards the adenoviruses of human origin, there may be preferably mentioned those classified in group C, in particular the adenoviruses of type 2 (Ad2), 5 (Ad5), 7 (Ad7) or 12 (Ad12). Among the various adenoviruses of animal origin, there may be preferably mentioned the adenoviruses of canine origin, and in particular all the strains of the CAV2 adenoviruses [manhattan or A26/61 strain (ATCC VR-800) for example]. Other adenoviruses of animal origin are cited in particular in application WO 94/26914 which is incorporated into the present by way of reference.

In a preferred embodiment of the invention, the recombinant adenovirus is a group C human adenovirus. More preferably, it is an Ad2 or Ad5 adenovirus.

The recombinant adenoviruses are produced in an encapsidation line, that is to say a cell line capable of complementing in trans one or more of the functions deficient in the recombinant adenoviral genome. One of these lines is, for example, the line 293 into which part of the adenovirus genome has been integrated. More precisely, the line 293 is an embryonic human kidney cell line containing the left end (about 11-12%) of the genome of the serotype 5 adenovirus (Ad5), comprising the left ITR, the encapsidation region, the E1 region, including E1a and E1b, the region encoding the pIX protein and part of the region encoding the pIVa2 protein. This line is capable of transcomplementing recombinant adenoviruses which are defective for the E1 region, that is to say which lack all or part of the E1 region, and of producing viral stocks having high titres. This line is also capable of producing, at a permissive temperature (32° C.), virus stocks comprising, in addition, the heat-sensitive E2 mutation. Other cell lines capable of complementing the E1 region have been described, based in particular on human lung carcinoma cells A549 (WO 94/28152) or on human retinoblasts (Hum. Gen. Ther. (1996) 215). Moreover, lines capable of transcomplementing several adenovirus functions have also been described. In particular, there may be mentioned lines complementing the E1 and E4 regions (Yeh et al., J. Virol. 70 (1996) 559; Cancer Gen. Ther. 2 (1995) 322; Krougliak et al., Hum. Gen. Ther. 6 (1995) 1575) and lines complementing the E1 and E2 regions (WO 94/28152, WO 95/02697 and WO 95/27071). The recombinant adenoviruses are usually produced by introducing the viral DNA into the encapsidation line, followed by lysis of the cells after about 2 to 3 days (the kinetics of the adenoviral cycle being 24 to 36 hours). After lysis of the cells, the recombinant viral particles are isolated by caesium chloride gradient centrifugation. Alternative methods have been described in application FR 96 08164 which is incorporated into the present by reference.

The cassette for expressing the therapeutic gene(s) may be inserted into different sites of the recombinant adenovirus genome, according to the techniques described in the prior art. It can first of all be inserted at the level of the El deletion. It can also be inserted at the level of the E3 region, as an addition or as a substitution of sequences. It can also be located at the level of the deleted E4 region. For the construction of vectors carrying two expression cassettes, one may be inserted at the level of the E1 region, the other at the level of the E3 or E4 region. Both cassettes can also be introduced at the level of the same region.

To carry out the present invention, the composition comprising the expression system can be formulated in various ways. It may be, in particular, isotonic sterile saline solutions (monosodium or disodium phosphate, sodium, potassium, calcium or magnesium chloride, and the like, or mixtures of such salts), or dry, in particular freeze-dried, compositions which, upon addition, depending on the case, of sterilized water or of physiological saline, allow the preparation of injectable solutions. Other excipients can also be used, such as, for example, stabilizing proteins (human serum albumin in particular: FR96/03074), poloxamer or a hydrogel. This hydrogel can be prepared from any biocompatible and non-cytotoxic (homo- or hetero-) polymer. Such polymers have, for example, been described in Application WO 93/08845. Some of them, such as in particular those obtained from ethylene oxide and/or propylene are commercially available. Moreover, when the expression system is composed of plasmid vectors, it may be advantageous to add to the composition one or more chemical or biochemical agents promoting the transfer of genes. In this regard, there may be mentioned more particularly cationic polymers of the polylysine (LKLK)n or (LKKL)n type as described in Application WO 95/21931, polyethyleneimine (WO 96/02655) and DEAE-dextran or cationic lipids or lipofectants. They possess the property of condensing DNA and of promoting its association with the cell membrane. Among these, there may be mentioned the lipopolyamines (lipofectamine, transfectam, as described in Application WO 95/18863 or WO 96/17823), various cationic or neutral lipids (DOTMA, DOGS, DOPE, and the like) as well as peptides of nuclear origin (WO 96/25508), optionally functionalized so as to target certain tissues. The preparation of a composition according to the invention using such a chemical vector is carried out according to any technique known to persons skilled in the art, generally by simply placing the various components in contact.

In a particularly preferred manner, the expression system used in the invention consists of a defective recombinant adenovirus encoding a neurotrophic factor. Still more particularly, the neurotrophic factor is NT3. For their use in the invention, the adenoviruses are advantageously formulated and administered in the form of doses of between 10⁴ and 10¹⁴ pfu, and preferably 10⁶ to 10¹⁰ pfu. The term pfu (“plaque forming unit”) corresponds to the infectivity of an adenovirus solution, and is determined by infecting an appropriate cell culture and measuring, generally after 15 days, the number of infected cell plaques. The techniques for determining the pfu titre of a viral solution are well documented in the literature. The examples below show quite remarkably that doses of 10⁹ and 10⁷ allow (i) an effective transfer of genes into sectioned neurons, (ii) a lasting expression of the transgene in the said neurons and (iii) restoration of axonal continuity.

The expression system can be introduced into the cuff in various ways, and in particular by means of syringes. Injection by means of microsyringes is preferred (Hamilton or Terumo microsyringe).

One of the particularly advantageous applications of the present invention is the stimulation of the regrowth of the peripheral nerves. This treatment can be applied in various pathological situations, in particular traumas or nerve degenerations. It can be applied to any surgically accessible nerve, and in particular to the radial nerves, the cubital nerves, the median nerves, the colateral nerves of the fingers and the inter-bone nerves, for the upper limbs, and to the sciatic nerves (diameter of about 1 cm at its birth) or crural nerves (diameter 6-7 mm), for the lower limbs.

Another particularly advantageous application of the invention is the restoration of nerve continuity at the level of the roots of the brachial plexus (diameter 5-6 mm) or within the spinal cord itself, following a trauma. There is currently no treatment for this type of lesion. The method of the invention makes it possible to perform a bridging between the section subjacent to a section of the marrow and the section superjacent thereto, so as to join the principal bundles and to regenerate nerve continuity. For these applications, the cuffs used preferably have an inner diameter which may be as high as 15 to 20 mm. In particular, for bridging a root extracted at the level of the brachial plexus, the diameter of the cuff corresponds to the diameter of the root.

The subject of the present invention is therefore also a product for the local and prolonged release of a neurotrophic substance at the level of a nerve lesion composed of a biocompatible cuff which makes it possible to join the parts above and below the lesions, into which a system for expressing a neurotrophic factor is introduced.

The present invention can be used to stimulate nerve regeneration in vivo both in animals and in humans. It can, in addition, be used, in animals, to study the properties of a new trophic factor (a new protein, a mutant, and the like). For that, an animal is subjected to a nerve section, and then a system for expressing the factor to be tested is introduced into a device according to the invention. The capacity of the said factor to restore nerve continuity is determined as indicated in the examples. This device makes it possible, in addition, to compare various factors, or to study synergistic associations of various factors.

The present invention will be described in greater detail with the aid of the following examples, which should be considered as illustrative and non-limiting.

Legend to the figures

FIG. 1: Description of the fitting, over a peripheral nerve, of a device according to the invention.

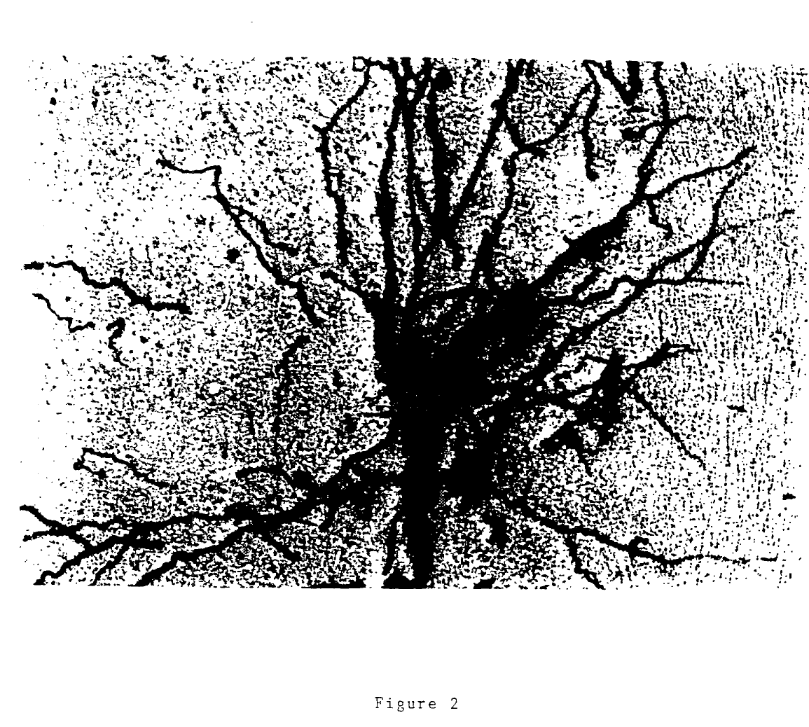

FIG. 2: Micrograph taken at the level of the sacrolumbar portion of the spinal cord and showing a high production of β-galactosidase (revealed with the X-Gal substrate) inside spinal motoneurons.

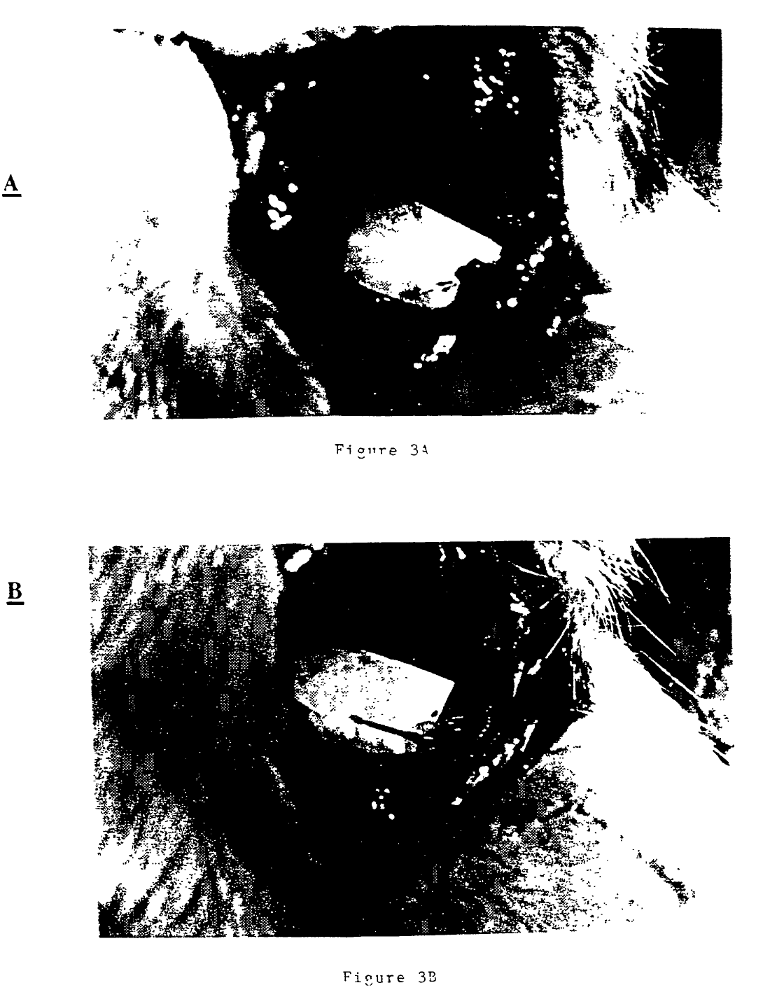

FIG. 3: Macroscopic appearance of the tissue regrowth on D12. (A) Example observed in the control animal. No tissue continuity is observed between the proximal and distal ends of the nerve repair. (B) Appearance of the contents of the stent in an animal which has received an injection of 10⁷ pfu Ad-NT3. It is possible to note the presence of a tissue link joining the proximal and distal ends of the nerve repair.

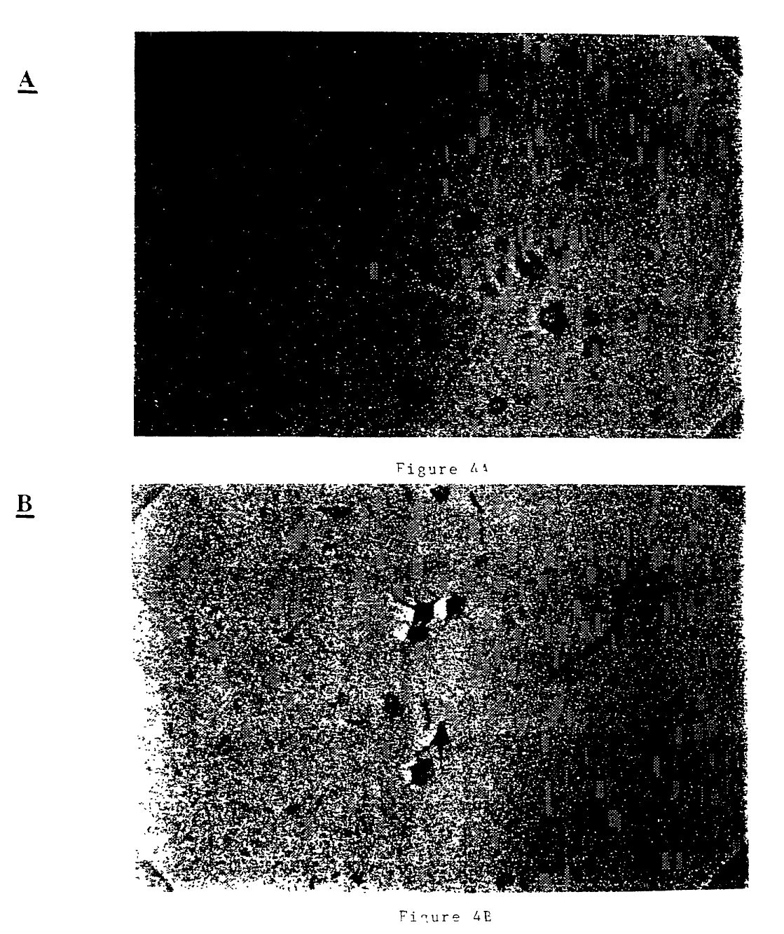

FIG. 4: Appearance of retrograde stains with HRP observed on D12. (A) In the control group, a few rare, weakly labelled, motoneurons are observed. (B) In the group treated with 10⁷ pfu Ad-NT3, a large number of strongly labelled spinal neurons are present.

FIG. 5: Description of the putting in place according to the invention of a bridging of the peripheral afferences subjacent to a lesion at the level of the healthy marrow superjacent to the said lesion.

FIG. 6: Description of the putting in place, in the spinal cord, of a device according to the invention.

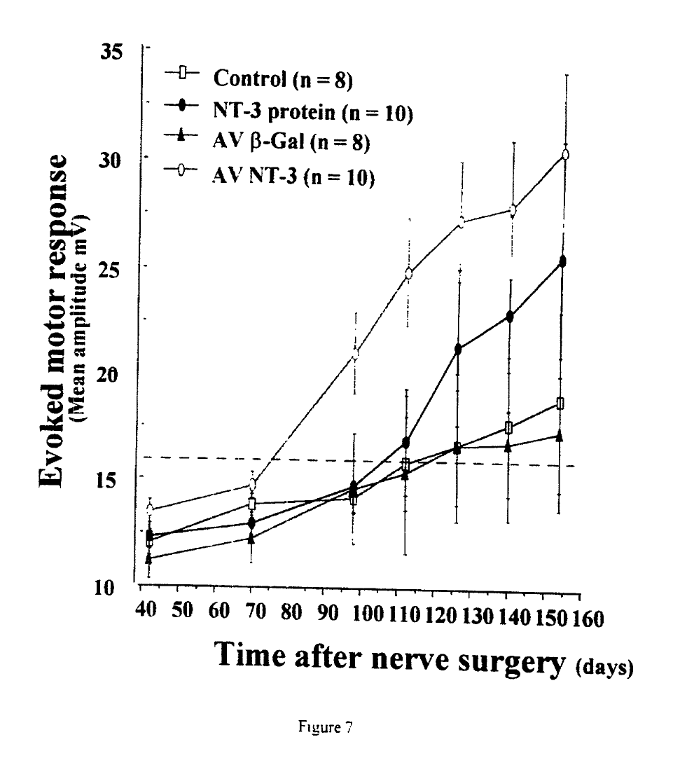

FIG. 7: Description of the motor response observed as a function of time after operating on the nerve and fitting the device according to the invention.

1. Methodology

1-1. Adenoviral vectors

As indicated above, the viral vectors, and in particular adenoviruses, constitute a particularly preferred embodiment of the invention.

The recombinant adenoviruses used were obtained by homologous recombination according to the techniques described in the prior art. Briefly, they are constructed in cells 293 by recombination between a linearized viral genome fragment (dl324) and a plasmid containing the left ITR, the encapsidation sequences, the transgene as well as its promoter and viral sequences allowing recombination. The viruses are amplified on cells 293. They are regularly repurified in the P3 in our laboratory. The viral genomes can also be prepared in a prokaryotic cell according to the technique described in Application WO 96/25506. The following viruses are more particularly used:

AD-βGal: Defective recombinant adenovirus derived from an Ad5 serotype comprising (i) a deletion of the E1 region at the level of which there is introduced an expression cassette comprising a nucleic acid encoding E. coli β-galactosidase under the control of the Rous sarcoma virus LTR promoter (designated RSV-LTR or RSV), and (ii) a deletion of the E3 region. The construction of this adenovirus has been described in Stratford-Perricaudet et al. (J. Clin. Invest. 90 (1992) 626).

Ad-NT3: Recombinant adenovirus of the Ad5 serotype comprising, inserted into its genome in place of the deleted E1 region, an NT3 expression cassette composed of the cDNA encoding NT3 under the control of a transcriptional promoter (in particular the RSV LTR). An alternative construction comprises an additional deletion in the E4 region, as described in Application WO 96/22378.

Ad-CNTF: Recombinant adenovirus of the Ad5 serotype comprising, inserted into its genome in place of the deleted E1 region, a CNTF expression cassette composed of the cDNA encoding CNTF under the control of a transcriptional promoter (in particular the RSV LTR). Details of the construction are given in Application WO 94/08026. An alternative construction comprises an additional deletion in the E4 region, as described in Application WO 96/22378.

Ad-GDNF: Recombinant adenovirus of the Ad5 serotype comprising, inserted into its genome in place of the deleted E1 region, a GDNF expression cassette composed of the cDNA encoding GDNF under the control of a transcriptional promoter (in particular the RSV LTR). Details of the construction are given in Application WO 95/26408. An alternative construction comprises an additional deletion in the E4 region, as described in Application WO 96/22378.

Ad-BDNF: Recombinant adenovirus of the Ad5 serotype comprising, inserted into its genome in place of the deleted E1 region, a BDNF expression cassette composed of the cDNA encoding BDNF under the control of a transcriptional promoter (in particular the RSV LTR). Details of the construction are given in Application WO 95/25804. An alternative construction comprises an additional deletion in the E4 region, as described in Application WO 96/22378.

Ad-FGFα: Recombinant adenovirus of the Ad5 serotype comprising, inserted into its genome in place of the deleted E1 region, an FGFα expression cassette composed of the cDNA encoding FGFα under the control of a transcriptional promoter (in particular the RSV LTR). Details of the construction are given in Application WO 95/25803. An alternative construction comprises an additional deletion in the E4 region, as described in Application WO 96/22378.

The functionality of the viruses constructed is checked by infecting fibroblasts in culture. The presence of the corresponding neurotrophic factor is analysed in the culture supernatant by ELISA and/or by testing for the trophic properties of this supernatant on neuronal primary cultures.

It is understood that other constructs derived from adenoviruses can be prepared and used within the framework of the invention, and in particular vectors carrying additional deletions and/or different promoters and/or encoding other neurotrophic factors.

1-2. Surgical protocol

The animals consisted of Sprague-Dawley male rats weighing 320-340 g (Iffa Credo—Les Oncins—France). Under general anaesthesia (intraperitoneal injection of pentobarbital 1 ml/kg—Sanofi Santé Animale), the skin of the right hind leg is incised at the level of the thigh, and the muscle planes are separated so as to reveal the right sciatic nerve. The nerve is sectioned halfway between the popliteal space and the separation of the sciatic nerve, and a 5 mm segment is removed (FIG. 1B). A tubular silicone prosthesis (14 mm in length, 1.47 mm in diameter, wall thickness: 0.23 mm—Silastic, Dow Corning Corporation, USA) is presented. The proximal end of the nerve is introduced into the tube and is held in place with the aid of a nylon 9/0 suture connecting the spinal cord and the nerve. A second suture between the tube and the nerve at the distal level is put in place so as to obtain a loss of substance of 10 mm (maximum distance for which a spontaneous peripheral nerve regeneration can be observed in rats under the experimental conditions used) (FIG. 1C). The leaktightness of the assembly at the proximal level is then obtained with the aid of a fibrin glue (Tissucol, Immuno AG, Vienna, Austria), before introducing into the stent, in contact with the proximal end of the nerve, 10 μl of the viral solution, or of isotonic saline solution for the control animals, with the aid of a microsyringe (FIG. 1D). The dead volume of the stent is filled with an isotonic saline solution (0.9% sodium chloride solution), before the distal end of the nerve is introduced, in its turn, into the tubular prosthesis, and the leaktightness of this end ensured by the fibrin glue (FIG. 1E). The muscle and skin planes are closed with the aid of a standard 6/0 and 4/0 nylon suture, respectively. The animals are placed in an individual cage, and maintained in a 12 h/12 h day/night cycle.

1-3. Control of axonal regrowth and retrograde staining of the spinal motoneurons by Horseradish Peroxidase (HRP)

Twelve days later, and after the animals have been placed under a general anaesthetic, the assembly is re-exposed and dissected from the surrounding adhering materials. The presence of tissue continuity is noted before the assembly is sectioned at 3 mm downstream of the proximal end of the original nerve section. The “stump” thus obtained is rinsed with isotonic saline solution, before being filled with a 30% (w/v) HRP solution (Sigma Chemical, St. Louis, Mo., USA). After incubating for 1 hour, this solution is removed, and the nerve ending rinsed with an isotonic saline solution before closing the muscle and skin planes and putting the animals back in their cages. Forty-eight hours later, the animals are reanaesthetized, and fixed, after rinsing with PBS, by intracardiac infusion of 3.6% glutaraldehyde. The spinal cords are then dissected, post-fixed for 3 hours in 3.6% glutaraldehyde, and placed in 30% (w/v) sucrose for 48 hours to 72 hours. The lumbosacral parts of the marrows are cut frozen into longitudinal serial sections 35 μm thick, and the presence of HRP revealed according to the conventional technique described by Mesulam (15), and using 3,3′,5,5′-tetramethylbenzidine.

1-4. Detection of β-galactosidase

The β-galactosidase activity was visualized using the X-Gal substrate (14). Briefly, longitudinal sections of the sacrolumbar marrow 100 μm thick are incubated for 18 h at 37° C. in PBS containing potassium hexacyanoferrate (4 mM), potassium ferricyanide (4 mM), X-Gal substrate (0.4 mg/ml), and magnesium chloride (4 mM). After incubation, the tissue sections are rinsed in PBS and then mounted in an aqueous medium (Gelatin-Glycerol).

2. Demonstration of retrograde transport of nonrepetitive adenoviruses by the axotomized spinal motoneurons, and verification of the expression of the transgene

This first study made it possible to demonstrate that axotomized neurons could perform a retrograde transport of adenoviral vectors and express a transgene over times which may be as long as 4 weeks. The vector (Adenovirus β-galactosidase described in 1-1.) was injected in an amount of 10⁹ pfu per tube, and the expression of its transgene tested at D4, D14 and 4 weeks.

At 4 days, a high expression of β-galactosidase was observed in the ventral horn of the sacrolumbar portion of the spinal cord corresponding to the roots innervating the sciatic nerve (FIG. 2). This high expression of the transgene by the spinal motoneurons was found at 14 days with in total a mean number of β-galactosidase positive cells of 63.2±33.6 cell., that is to say an efficiency of infection of the order of 12.25% of the total number of spinal neurons innervating the sciatic nerve. The presence of a β-galactosidase activity in the sacrolumbar region of the spinal cord was detected up to 4 weeks with a decreasing labelling intensity (Table I).

3. Test of the effect of a vector encoding a neurotrophin (NT3) on the axonal regrowth of rat sciatic nerve through a loss of substance of 10 mm

Following these results showing the possibility of using a gene therapy type system in vivo to stimulate nerve regrowth after axotomy, Following these results showing the possibility of using a gene therapy type system in vivo to stimulate nerve regrowth after axotomy, we tested the effect of an adenovirus carrying a transgene encoding neurotrophin-3 (Ad-NT3 described in 1-1.) on peripheral nerve regeneration. The results obtained 12 days after carrying out the axotomy and repair of the nerve with a guide stent made of Silastic having received 10⁷ pfu of vector, or an isotonic saline solution, show that tissue continuity is observed only in the group of animals treated with Ad-NT3 (FIG. 3, Table II). Analysis by retrograde staining with HRP of the number of spinal motoneurons which have regenerated an axon through the guide stent indicates that this tissue continuity consists of nerve regrowth, with a mean of 182.3±76.5 HRP-positive neurons against 24.25±42.7 HRP-positive neurons in the control group (FIG. 4, Table II).

These results make it possible to conclude that a device according to the invention, using replication-deficient adenovirus vectors carrying genes encoding neurotrophic factors, can be used to promote axonal regrowth which is both central and peripheral.

4. Comparison of the functional revival after section of a peripheral nerve in rats

A lesion was performed at the level of the sciatic nerve in adult rats in order to create a loss of substance of at least 10 mm. The proximal and distal parts of the lesion were joined by means of a device according to the invention (silicone tube, 14 mm in length, 1.47 mm in internal diameter—Silastic) into which there has been introduced either a saline solution, or AV-RSVβgal (10⁷ pfu in 10 μl), or AV-RSVNT₃ (10⁷ pfu in 10 μl), or alternatively the NT3 protein. The functional revival was measured by electromyography: the motor response in the gastronemius muscle was recorded every two weeks (FIG. 7).

A functional revival was observed in the group treated with AV-RSVNT₃ compared with the other groups. This increase was statistically significant compared, over time, with the AV-RSVβgal group after day 112, and from day 70 to day 112 with the group rNT3. An electromyographic analysis of the individual profiles shows that treatment with AV-RSVNT₃ increases the probability for a given animal to initiate the regrowth of the nerve. However, the regrowth level is not modified when regrowth has started.

These results therefore suggest that the transfer of a gene encoding a neurotrophic factor by means of the technique described in the present invention is effective for increasing functional revival after section of a peripheral nerve.

### BIBLIOGRAPHIC REFERENCES

1—Archibald et al.—J. Comp. Neurol. 1991, 306, 685-696.

2—Lundborg et al.—J. Neuropathol. Exp. Neurol. 1982, 41, 412-422.

3—Lundborg et al.—Exp. Neurol. 1982, 76, 361-375.

4—Seckel et al.—Plast. Reconstr. Surg. 1986, 78, 793-798.

5—Lundborg et al.—Scand. J. Plast. Reconstr. Surg. Hand Surg. 1991, 25, 79-82.

6—Lundborg et al.—J. Hand Surg. 1994, 19, 273-276.

7—Cordeiro et al.—Plast. Reconstr. Surg. 1989, 13, 183-198.

8—Aebisher et al.—J. Neurosci. Res. 1989, 23, 282-289.

9—Laquerriere et al.—Microsurg. 1994, 15, 203-210.

10—Derby et al.—Exp. Neurol. 1993, 119, 176-191.

11—Sahenk et al.—Brain Res. 1994, 655, 246-250.

12—Glazner et al.—Neurosci. 1993, 54, 791-797.

13—Van der Vliet et al., 1975

14—Finiels et al.—Neuroreport, 1995, 7, 373-378.

15—Mesulam—J. Histochem. Cytochem. 1978, 26, 106-117.

16—Henderson, Adv. Neurol. 68 (1995) 235

17—Thoenen (Trends in NeuroSci. 14 (1991) 165

18—Lindsay in Neurotrophic Factors, Ed, (1993) 257, Academic Press

19—Maisonpierre et al., Genomics 10 (1991) 558

20—Leibrock et al., Nature 341 (1989) 149

21—Henderson et al., Nature 363, 266-270 (1993)

22—Hohn et al., Nature 344 (1990) 339

23—L.-F. Lin et al., Science, 260, 1130-1132 (1993)

TABLE I
Determination of the efficiency of infection
of the axotomized motoneurons by an adenoviral vector
encoding for β-galactosidase

| | | Number of | Number of | Total |
|---|---|---|---|---|
| | | highly | labelled | number of |
| | | labelled | cellular | labelled |
| Period | Animals | neurons | bodies | neurons |
| D4 | 1 | 10 | 12 | 22 |
| | 2 | 36 | 55 | 91 |
| | 3 | 17 | 31 | 48 |
| D14 | 1 | 16 | 24 | 40 |
| | 2 | 59 | 48 | 107 |
| | 3 | 24 | 6 | 30 |
| 4 week | | | | Diffuse |
| | | | | labelling |

TABLE II
Effect of the injection of an Ad-NT3 on axonal regrowth at D12

| | | | | Number of |
|---|---|---|---|---|
| | | | | HRP- |
| | | | Tissue | positive |
| Group | Animal | Period | continuity | neurons |
| Control | T01 | D12 | + | 0 |
| | T02 | D12 | − | 9 |
| | T03 | D12 | − | 88 |
| | T05 | D13 | − | 0 |
| Ad-NT3 | N71 | D12 | +++ | 182 |
| 10⁷ pfu | N72 | D12 | +++ | 106 |
| | N73 | D12 | +++ | N.D.* |
| | N75 | D14 | +++ | 259 |

N.D.: not determined
* Animal died during infusion.

1. Device for stimulating nerve regeneration, comprising a biocompatible cuff into which a system for expressing a neurotrophic factor is introduced.

2. Kit for stimulating nerve regeneration, comprising, on the one hand, a biocompatible cuff, and, on the other hand, a composition comprising a system for expressing a neurotrophic factor.

3. Use, for the preparation of a composition intended to stimulate nerve regeneration, of a biocompatible cuff into which a system for expressing a neurotrophic factor is introduced.

4. Use according to claim 3, for the preparation of a composition intended to stimulate the regeneration of the peripheral nerves.

5. Use according to claim 3, for the preparation of a composition intended to stimulate axonal regeneration in the spinal cord.

6. Use, for the preparation of a composition intended for the treatment of traumatic lesions of the nervous system, of a biocompatible cuff into which a system for expressing a neurotrophic factor is introduced.

7. Product for the local and prolonged release of a neurotrophic substance at the level of a nerve lesion composed of a biocompatible cuff which makes it possible to join the parts above and below the lesions, into which a system for expressing a neurotrophic factor is introduced.

8. Use according to one of claims 3 to 6, characterized in that the cuff consists of a tubular support made of nontoxic and biocompatible materials.

9. Use according to one of claims 3 to 6, characterized in that the first nerve section is introduced into one end of the cuff where it is kept in place by a suture and/or glue, the expression system is introduced into the cuff, and then the second section of the nerve is inserted into the second end of the cuff where it is held by suture and/or glue.

10. Use according to one of claims 3 to 6, characterized in that the expression system consists of a vector comprising a nucleic acid encoding the said neurotrophic factor.

11. Use according to claim 10, characterized in that the vector is a viral vector.

12. Use according to claim 11, characterized in that the viral vector is an adenoviral vector.

13. Use according to claim 10, characterized in that the neurotrophic factor is chosen from the factors of the neurotrophin, neurokine, beta-TGF, FGF and IGF family.

14. Use according to claim 13, characterized in that the neurotrophic factor is chosen from BDNF, GDNF, CNTF, NT3, FGFα and IGF-I.
